(19) United States
(12) Patent Application Publication
(10) Pub. No.: US 2011/0009967 A1
(43) Pub. Date: Jan. 13, 2011

(54) Intervertebral Spinal Implant and Method of Making the Same

(76) Inventors: Theodore I. Malinin, Key Biscayne, FL (US); H. Thomas Temple, Miami, FL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(21) Appl. No.: 12/874,804

(22) Filed: Sep. 2, 2010

Related U.S. Application Data
(62) Division of application No. 11/775,656, filed on Jul. 10, 2007.
Application No. 11/775,656 is a continuation of application No. 12/823,811, filed on Jun. 25, 2010.
Application No. 12/823,811 is a continuation-in-part of application No. 11/775,656, filed on Jul. 10, 2007.

(51) Int. Cl. A61F 2/44

(52) U.S. Cl. 623/17.11

(57) ABSTRACT

An intervertebral implant for replacing a damage intervertebral disk within the spinal column can include a generally flat body having opposing surfaces. To promote bone growth and fusion of the adjacent vertebrae together, in an aspect, the implant can be provided with osteogenic or medicinal material received into one or more apertures disposed into the surfaces of the implant. To better retain the osteogenic material, the apertures can be disposed on a non-perpendicular angle with respect to the implant and/or can be tapered. In another aspect, the implant can include a plurality of grooves disposed across a surface to reduce slipping of the inserted implant between the adjacent vertebrae. In a further aspect, the implant can be formed by cutting a plurality of implants parallel along the diaphysis of a long bone. In another aspect, the implant can be generally as a bow or question mark.

### CROSS REFERENCE TO RELATED APPLICATIONS

This patent application is a divisional of copending U.S. patent application Ser. No. 11/775,656, filed Jul. 10, 2007. This patent application is a continuing application of copending U.S. patent application Ser. No. 12/823,811, filed Jun. 25, 2010, which is a continuation-in-part of U.S. patent application Ser. No. 11/775,656, filed Jul. 10, 2007.

### BACKGROUND OF THE INVENTION

In humans and other vertebrate animals, the spinal column is made of individual bones or vertebrae that are aligned together and extend along the center of an individual's back. Importantly, the spinal column provides a protective channel for the spinal cord of the central nervous system and supports an individual's weight and posture while enabling a wide range of motion of the upper body. The vertebrate are movably joined at facet joints and, in humans in particular, can be arranged in regions including the cervical region corresponding to the neck, the thoracic region corresponding to the chest, and the lumbar region corresponding to the lower back. The arrangement of vertebrae within the regions can provide the familiar curves and arches of the spinal column. To enable bending, twisting and rotating of the upper body, the individual vertebrae are spaced apart by intervertebral disks. The intervertebral disks are made of a tough, fibrous connective tissue that rings around and surrounds a thick, jelly-like material at the center of each disk. The disks act to dampen shock transmitted along the spinal column and to enable motion.

Intervertebral disks may become damaged or degenerate overtime, due to disease, or due to abrupt injury such that it may become medically necessary or beneficial to surgically remove the damaged disk. To maintain the intervertebral spacing between two adjacent vertebrae from which a disk has been removed, it is known to insert spinal or intervertebral implants into the space. The intervertebral implant preferably promotes bone growth to fuse the adjacent vertebrae across the disk space. A variety of materials, sizes, shapes, and insertion techniques have been suggested for providing and inserting intervertebral implants. For example, it is well known to shape the implants as cylindrical dowels that can be inserted between the vertebrae. In some instances, the implant can be formed of a biocompatible material such as metal or ceramic or can be formed from actual bone tissue harvested from a donor bone. Desirably, the material, size and shape of the implant are selected for ease of implantation, maintenance of the proper spinal curvature, and to provide the necessary biomechanical strength to support the spinal column.

In some instances, screws, braces or fixtures can be utilized to maintain alignment of the spinal column and implant during recovery and fusion of the adjacent vertebrae. In other instances, it may be desirable to incorporate osteogenic material with the intervertebral insert to promote bone tissue growth and fusion of the adjacent columns. Accordingly, there exists a need for an intervertebral spinal implant that can maintain the intervertebral space between and enable rapid fusion of adjacent vertebrae. There exist a further need for a intervertebral implant that is biologically active and biomechanically strong and that can maintain and support the existing curvature of the spinal column. Additionally, the intervertebral implant should remain stable and not be prone to slippage.

### BRIEF SUMMARY OF THE INVENTION

The invention provides an intervertebral spinal implant for maintaining intervertebral spacing between and promoting the fusion together of two adjacent vertebrae. In an aspect, the intervertebral implant can have a generally flat body with a first surface and an opposing second surface that is sized and shaped for insertion into the intervertebral space. Disposed into the body can be at least one aperture that can be formed to receive osteogenic or similar medicinal material that promotes bone growth between the vertebrate to fuse those vertebrate together. To optimize retention of the osteogenic material within the body during manipulation of the implant, the aperture in some embodiments can be disposed on a non-perpendicular angle into the first surface of the body. In other embodiments, the aperture can taper or be conically shaped as it extends from the first surface toward the second surface of the body. The tapering of the aperture can be in addition to or besides disposing the apertures on non-perpendicular angles. Another advantage of disposing the osteogenic material receiving aperture on a non-perpendicular angle or on a taper is that the material will tend not to shake or fall loose from the aperture. Another advantage is that the non-perpendicular or tapered apertures can accommodate more osteogenic material.

In another aspect of the invention, an intervertebral implant having a flat body with first and second opposing surfaces can have disposed into at least one surface a plurality of grooves. The grooves can have any suitable shape or pattern, but preferably have a gull-wing shape. To provide the gull-wing shape, the grooves can have a first curve and a second curve that intersect together approximately mid-width of the implant. The gull-wing shaped grooves can retain osteogenic or other medicinal material and can allow for ingrowth of the host bone. In various embodiments, the intervertebral implant can have gull-wing shaped grooves on both the first and second surface and further can include one or more osteogenic material receiving apertures of the above described kind. Another advantage of disposing the gull-wing shaped grooves across a surface of the implant is that grooves provide traction where the implant surface meets the vertebrae thereby preventing slipping or movement of the implant.

In another aspect of the invention, an intervertebral implant having a flat body and first and second opposing surfaces can be formed from the elongated diaphysis or shaft portion of a long donor bone. To form the implant, a plurality of outlines, each of the first surface, are cut or otherwise disposed directly into the outer surface of the bone tissue such that the plurality of outlines are arranged axially along the diaphysis. Accordingly, one surface of the implant corresponds to the outer surface of the diaphysis of the donor bone. This is in contrast to prior art methods, in which allografts or spinal implants are typically formed by disposing cuts perpendicularly into the diaphysis. An advantage of preparing the implants by cutting into the diaphysis parallel rather than perpendicular to its long axis is to conserve donor bone by enabling larger and more implants to be formed from a given bone.

In another aspect of the invention, the intervertebral implant can have a generally flat body generally shaped overall as a question mark. The question-mark shape can be provided by having a peripheral surface of the body include a straight first edge, a curved second edge extending away from the first edge, and a cutout formed into the first edge. In various embodiments, the cutout can receive osteogenic or other medicinal material. An advantage of forming the implant with a question-mark shape is that such a shape helps to fill the entire inter-vertebral.

Accordingly, an advantage of the inventive intervertebral implant is that it provides strong biomechanical support to the spinal column. Another advantage is that the intervertebral implant can retain osteogenic material for promoting fusion of adjacent vertebrae. A related advantage is that the intervertebral implant can include curved grooves of a specific shape to prevent slipping of the implant from between adjacent vertebrae. Yet another advantage is that the intervertebral implant can be shaped to promote and maintain the lordotic curve of the lumbar region in the spine. These and related advantages and features of the invention will become apparent upon review of the following drawing and

### BRIEF DESCRIPTION OF THE DRAWINGS

FIG. 1 is a top perspective view of an intervertebral spinal implant having a general “D”-shaped outline and a plurality of apertures disposed on a non-perpendicular angle therein.

FIG. 2 is a side elevational view of the intervertebral implant of FIG. 1 showing the non-perpendicular angle at which the apertures are disposed there through.

FIG. 3 is a schematic diagram illustrating one method of inserting the intervertebral implant into an intervertebral space of the spinal column.

FIG. 4 is a top plane view of another embodiment of an intervertebral implant having a “D”-shaped outline and a plurality of tapering apertures disposed on a non-perpendicular angle therein.

FIG. 5 is a side elevational view of the intervertebral implant illustrating the non-perpendicular angle that the tapered shape apertures are disposed along.

FIG. 6 is a bottom plan view of the intervertebral implant of FIG. 4.

FIG. 7 is a top plane view of another embodiment of an intervertebral implant having an elongated “D”-shape having a plurality of aperture disposed therein on various different non-perpendicular angles.

FIG. 8 is a top perspective view of another embodiment of an intervertebral implant having a plurality of non-perpendicular apertures and plurality of grooves disposed into a surface thereof, the grooves each having a gull-wing shape, the apertures and grooves retaining an osteogeneric material.

FIG. 9 is a perspective view of a diaphysis or shaft of an elongated donor bone having the outline of a plurality of intervertebral implants disposed therein in accordance with an aspect of the invention.

FIG. 10 is a top perspective view of an intervertebral implant having a question-mark shape.

### DETAILED DESCRIPTION OF THE EMBODIMENTS

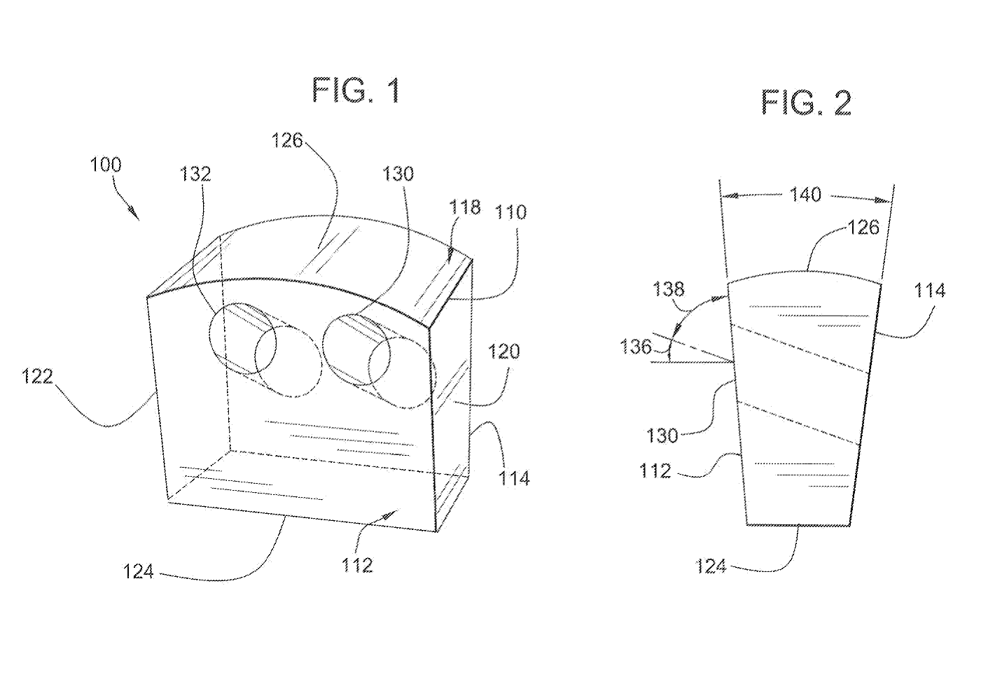

Now referring to the drawings, wherein like numbers refer to like elements, there is illustrated in FIGS. 1 and 2 an intervertebral implant 100 that can replace a damaged or ruptured intervertebral disk within the spinal column. The generally solid implant 100 can have a block-like shape including a generally flat body 110 including a first surface 112, an opposing second surface 114 and a peripheral surface 118 extending between the opposed first and second surfaces. In the illustrated embodiment, the intervertebral implant 100 can have a “D” shape in which the peripheral surface 118 further includes a first straight lateral edge 120, a second straight lateral edge 122 parallel to and spaced apart from the first lateral edge, and a third straight edge 124 extending between the first and second lateral edges. The peripheral surface further includes a curved edge 126 extending between the first and second edges 120, 122 and directed away from the third edge 124. As can be appreciated, the four edges of the peripheral surface generally provide the block-like shape to the intervertebral implant 100.

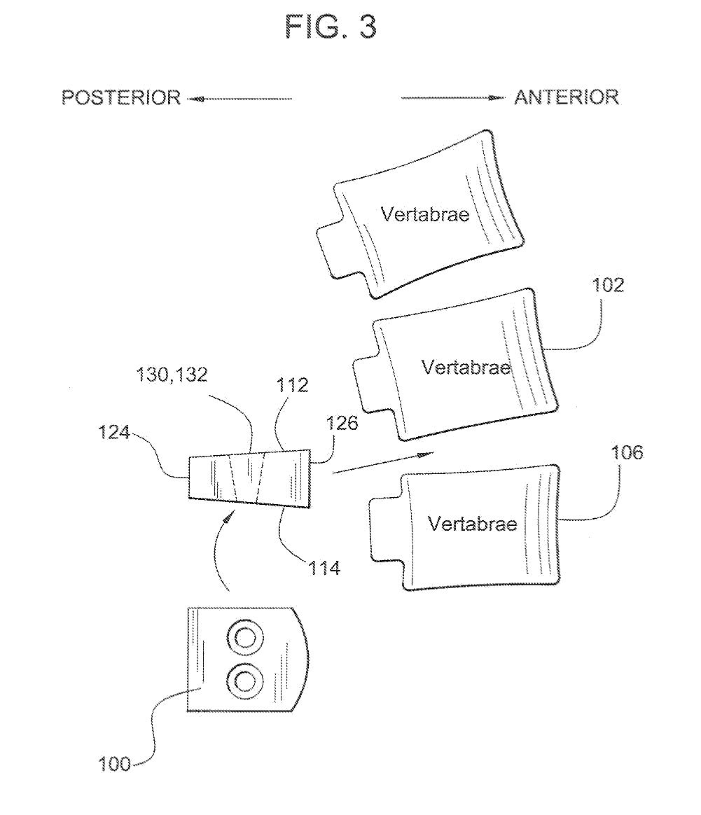

Replacement of a disk with the intervertebral implant 100 can be illustrated with reference to FIG. 3. First, the implant 100 is aligned between adjacent vertebrae including a upper vertebrae 102 and a corresponding lower vertebrae 106. The vertebrae can be separated by appropriate manipulation of the spinal column and the damaged intervertebral disk removed. The implant 100 can be aligned so that the curved fourth edge 126 is directed towards the anterior as indicated. Moreover, the upper surface 112 of the implant is oriented toward the upper vertebrae 104 and the lower surface 114 is oriented toward the lower vertebrae 106. The implant 100 is then inserted into the intervertebral space between the adjacent vertebrae and the upper and lower vertebrae can come to rest adjacent the respective upper and lower surfaces 112, 114. Preferably, the intervertebral implant 100 can assume a majority of the intervertebral space vacated by the removed disk. Because the implant is substantially solid, it can provide sufficient biomechanical strength for supporting the spinal column. While FIG. 3 illustrates insertion from the posterior direction, in other embodiments, insertion can occur from the anterior or other suitable direction.

Referring back to FIGS. 1 and 2, the intervertebral implant 100 can have any size suitably selected for the particular disk the implant is intended to replace. By way of example only, the implant can have an average width between the first lateral edge 120 and the second lateral edge of about 3.0 cm. Moreover, the implant 100 can have an average length between the third straight edge 124 and the apex of the fourth curved edge 126 of between about 1.3 cm to about 2.5 cm. The thickness of the implant 100 between the first surface 112 and the second surface 114 can be between about 0.5 cm and 2.0 cm. In other embodiments, these dimensions can be selected to traverse the majority of the length and width of the intervertebral space such that surface area of contact between upper and lower vertebrae is about 68 square cm and can be 200 square cm or more.

The material of the intervertebral implant 100 can be selected from any suitable biocompatible material having the desired biomechanical strength, immune acceptance and toxicity characteristics. For example, the material can be selected from a biologically compatible metal such as titanium, cobalt or chrome steel, gold alloys, stainless steel or similar metals. In other embodiments, the material can be selected from a synthetic, biologically active or bio-absorbable material such as calcium, sulfate, polyglycolic acid, hydroxyapatite, porous ceramics, apatitic bone cement, calcium phosphate, hydroxyproline, hydroxyapatite cement, and methylmetacrylate. In certain embodiments, the implant material can be selected from bio-active or bio-inactive bone tissue. For example, the bone tissue can be primarily cortical tissue such as typically found on the hard, solid outer surface of a donor bone. The bone tissue can also be primarily spongy cancellous bone typically found in the interior of thicker bones. When used in intervertebral implants, cortical bone may be desirable for its biomechanical strength properties but cancellous bone may be desirable for its ability to promote vascularization and new bone growth to fuse the adjacent vertebrae together. Accordingly, to increase biomechanical strength, the implant can be made from 90% cortical bone while to promote bone growth, the implant can be made from about 60% to 98% cancellous bone. When bone tissue is taken from a donor bone to form the intervertebral implant, the donor bone can be selected such that the resulting intervertebral implant can be an allograft (same animal species) or a xenograft (different animal species).

To address the above paradox, the intervertebral implant 100 can be configured to have an implant body 110 of relatively harder material and to retain or include a bioactive osteogenic material or similar medicinal material. To carry the osteogenic material, the implant body 110 can have a first aperture 130 and a second aperture 132 disposed into the first surface 112 and directed toward the second surface 114. The apertures 130, 132 may or may not traverse the entire thickness of the implant body 110. Moreover, in the illustrated embodiment, the apertures can be circular in cross-section but in other embodiment may have different shapes. The osteogenic or medicinal material can be placed or packed into the apertures 130, 132 prior to insertion of the implant into the intervertebral space. As can be appreciated with respect to FIG. 3, because the apertures 130, 132 are disposed through the first and second surfaces 112, 114 that are placed physically adjacent the upper and lower vertebrae, the osteogenic material can contact the vertebrae to promote bone grown and fusion.

The osteogenic material can be selected from any suitable material that helps promote bone growth and thereby speed fusion of adjacent vertebrae. For example, the osteogenic material can be selected from non-de-mineralized particular bone material, de-mineralized bone matrix, partially de-mineralized bone material, partially de-calcified bone material, AAA bone graft, or osteogenic growth factors including BMP. Moreover, the osteogenic material can be provided as a particulate, a jelly, a paste or a putty.

To optimize retention of the osteogenic and/or medicinal material in the aperture during handling and insertion of the implant 100, the apertures 130, 132 can be disposed on a non-perpendicular angle into the first surface 112 and towards the opposing second surface 114. Specifically, as best illustrated in FIG. 2, the axis line of the aperture 130 can be disposed on an angle 136 with respect to an imaginary line extending normally from the plane of the first surface 112. Angle 136 can be any suitable angle, for example, about 20° . Accordingly, the aperture 130 is disposed on a complementary angle with respect to the plane of the first surface 112 itself. An advantage of the non-perpendicular apertures 130, 132 is that they can better retain the osteogenic material by preventing the material from shaking loose or falling out of the insert and for accommodating a larger volume of osteogenic material. For example, non-perpendicular apertures have more exposed surface area to frictionally contact the osteogenic material than would perpendicular apertures. Additionally, the non-perpendicular apertures 130, 132 ensure that the retained material is not directly acted upon by gravity when the implant 100 is laid on either surface 112, 114 but is instead supported by the corresponding material of the body 110 delineating the channel of the apertures. Another advantage of disposing the apertures 130, 132 on a non-perpendicular angle is that the apertures define a greater volume through the body 110 and can therefore retain more osteogenic material.

Continuing to refer to FIG. 2, the opposing first and second surfaces 112, 114 of the intervertebral implant 100 need not be parallel with each other but instead can diverge on a given angle 140 from each other with respect to the straight third edge 124. The angle 140 is preferably selected so that the overall shape of the intervertebral implant 100 remains substantially flat. For example, the angle 140 can be about 9° . An advantage of angling the first and second surfaces 112, 114 with respect to each other can be appreciated with respect to FIG. 3. When the intervertebral implant 100 is inserted into the intervertebral space with the resulting thicker fourth edge 126 oriented toward the anterior of the spinal column and the relatively thinner third edge 124 oriented toward the posterior, it will be appreciated that the adjacent vertebrae 102, 106 are likewise maintained at an angle with respect to each other within the spinal column. Angling the vertebrae helps maintain the lordotic curve of the spinal column in the lumbar region of the lower back. Otherwise, loss of the lordotic curve may result in imbalance problems occurring to patient, pain, loss of the motion, and improper fusing of the adjacent vertebrae.

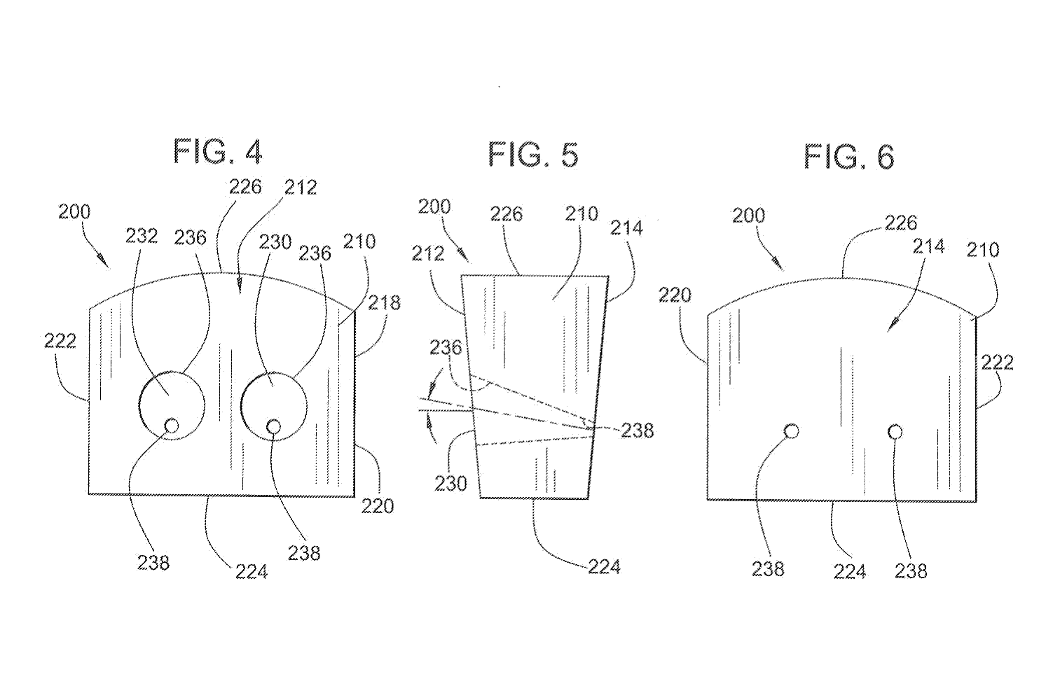

Referring now to FIGS. 4-6, there is illustrated another embodiment of an intervertebral implant 200 for replacing a damaged disk between two adjacent vertebrae. The illustrated intervertebral implant 200 again has a relatively solid implant body 210 having a block-like shape with a first surface 212, an opposing second surface 214, and a peripheral surface 218 extending there between. In the illustrated embodiment, the peripheral surface 218 again outlines or provides a “D” shape to the implant body 210. Particularly, the peripheral surface 218 can include a first straight lateral edge 220, a parallel and spaced apart second straight lateral edge 222, and a third straight edge 224 extending between the first and second laterals edges. The peripheral edge 218 also includes a fourth curved edge 226 extending between the first and second straight edge 220, 222. The fourth curved edge 226 is spaced apart and curves away from the third straight edge 224. Of course, in other embodiments, the implant body 210 can have other suitable shapes. Additionally, the intervertebral implant 200 can have dimensions corresponding to those provided above.

To retain the osteogenic or medicinal material, the intervertebral implant 200 can include a first aperture 230 and a second aperture disposed into the first surface 212 and directed toward the second surface 214. While in the illustrated embodiment, the apertures are disposed entirely through the implant body 210, it will be appreciated that in other embodiments, the apertures may terminate prior to the second surface 214. To optimize retention, the apertures 230, 232 can have a tapered or conical shape as they are disposed through the implant body 210 from the first surface 212 toward the second surface 214. Specifically, the circular apertures 230, 232 can form a larger diameter hole 236 proximate the first surface 212 and a smaller diameter hole proximate 238 the second surface 214. Tapering the apertures cause more surface area of the implant body 210 to frictionally contact the osteogenic material, thereby preventing the material from shaking or falling loose of the intervertebral implant 200. Additionally, the smaller diameter hole 238 restricts the osteogenic material from passing out the apertures 230, 232 via the second surface 214. In another embodiment, instead of tapering the aperture, the aperture can be formed as a counterbore having a first section of a larger diameter disposed into the first surface and a second section of a smaller diameter disposed into the second surface. Accordingly, in the present embodiment, the intervertebral implant 200 is inserted into the intervertebral space such that the second surface 214 is oriented toward the lower vertebrae.

To further improve osteogenic material retention, the tapered aperture 230, 232 can also be disposed into the first surface 212 on a non-perpendicular angle. Specifically, as illustrated with respect to FIG. 5, the axis line of the aperture 230 is offset with respect to an imaginary line extending perpendicularly from the plane of the first surface 212 by an angle 240. Angle 240 can be any given angle including, for example, 20° . Disposing the apertures 230, 232 on a non-perpendicular angle can realize the benefits mentioned above with respect to FIGS. 1 and 2.

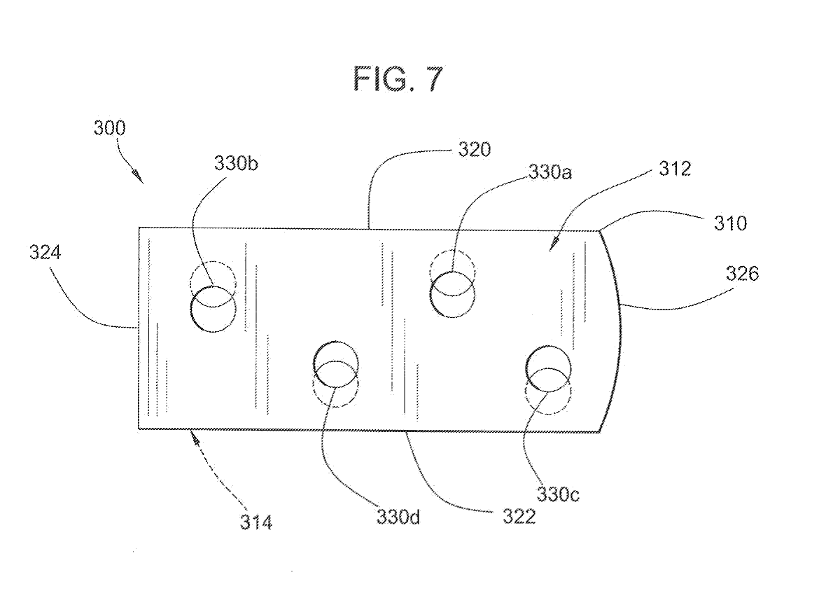

Referring now to FIG. 7, there is illustrated another embodiment of an elongated intervertebral implant 300 for replacement of a damaged spinal disk between adjacent vertebrae. The intervertebral implant 300 has a solid, block-like body 310 of any of the foregoing materials such as bone tissue, biocompatible metals, and biocompatible synthetic materials. The elongated intervertebral implant 300 includes a first surface 312, an opposing second surface 314, and a peripheral surface 318 extending there between. To provide the elongated shape to the body 310, the parallel first and second straight lateral edges 320, 322 are substantially longer than the third straight edge 324 extending between the first and second edges 320, 322. By elongating the shape the implant can be configured to assume the majority of the intervertebral space between even the largest or longest of the vertebrae, such as those associated with the lumbar region. Again, the intervertebral implant 300 can have a “D” shape provided by a curved fourth edge 326 located opposite the third straight edge 324 or, in other embodiments, can have other suitable shapes.

To retain the osteogenic or medicinal material, a plurality of apertures 330 can be disposed on non-perpendicular angles to the first surface 312 of the intervertebral implant 300. In particular, four separate apertures 330a, 330b, 330c, and 330d can be disposed along the elongated axis of the implant 300. The first two apertures 330a, 330b, are disposed near to the first lateral edge 312 and further are angled toward the first lateral edge. The second two apertures 330c, 330d are disposed near to the second side 314 and likewise are angled toward that second edge. Accordingly, in the illustrated embodiment the apertures 330 are not parallel to each other. As can be appreciated, all the plurality of apertures 330 could also be tapered, or only a portion of the plurality of apertures could be tapered.

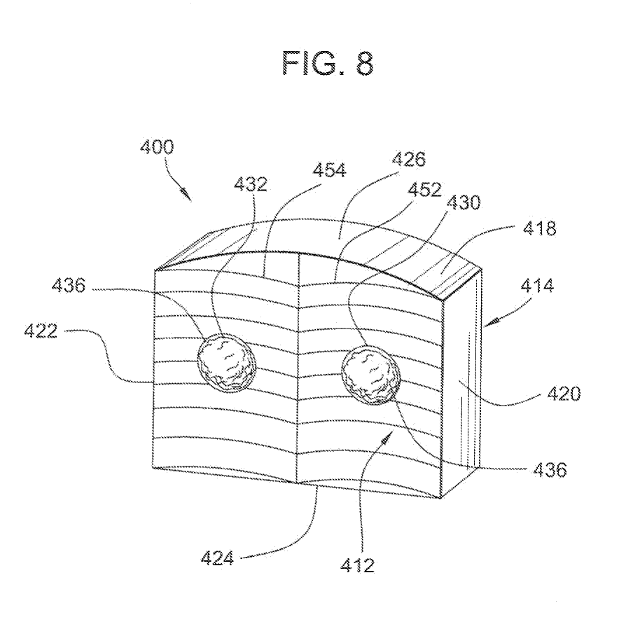

Referring to FIG. 8, in another aspect of the invention, the intervertebral implant 400 can include a plurality of grooves 450, as described below, disposed into one or more of its surfaces. The illustrated intervertebral implant 400 again can have a flat, block-like implant body 410 including a first surface 412 and an opposing second surface 414 interconnected by a peripheral surface 418. The peripheral surface can include a first straight lateral edge 420, a parallel second straight lateral edge 422, a third straight lateral edge 424 extending between the first and second edges 424, and a curved fourth edge 426 space apart and directed away from the third straight edge 424. The implant body can be made from any of the aforementioned suitable materials. Moreover, the intervertebral implant can include first and second apertures 430, 432 disposed therein that can be angled and/or tapered and as illustrated can retain osteogenic material 436.

In the illustrated embodiment, the plurality of grooves 450 are disposed across the first surface 412 and extend between the parallel first and second lateral edges 420, 422. In other embodiments, the grooves can be oriented in other directions to facilitate different insertion methods. The grooves can be disposed into the first surface any suitable depth, but should not thoroughly alter the strength or integrity of the intervertebral implant. For example, the depth of the grooves into the first surface can be about 1-2 mm and the spacing in between adjacent grooves in the plurality can be about 1 mm. Moreover, any number of grooves 450 can be in the plurality, and preferably the plurality of grooves are arranged in a gull-wing pattern. Specifically, the grooves 450 are parallel to each other and extend between the first and second adjacent lateral edges. To form the gull-wing pattern, the grooves 450 can each include a first curve 452 located proximate to the first lateral edge 420 and a second curve 454 located proximate to the second lateral edge 422. Both the first and second curves 452, 454 are directed towards the fourth curved edge of the implant, and the curves of each groove can intersect approximately mid-width between the first and second lateral edges. The grooves can help maintain position of the intervertebral implant sandwiched between the adjacent vertebrae by providing or encouraging friction between the surfaces of the implant and vertebrae that prevents slipping. In this regard, the gull-wing shaped grooves can be oriented so that the intersection between curves is in the direction of the intervertebral space into which the implant is inserted. This reduces the likelihood that the implant will become displaced before the implant and the vertebrae fuse together. Additionally, as illustrated, the plurality of grooves can also received and retain additional osteogenic material 436. Because the grooves extend across the surfaces 412, 414 of the implants 400, the osteogenic material is advantageously spread across the implant-vertebrae interface.

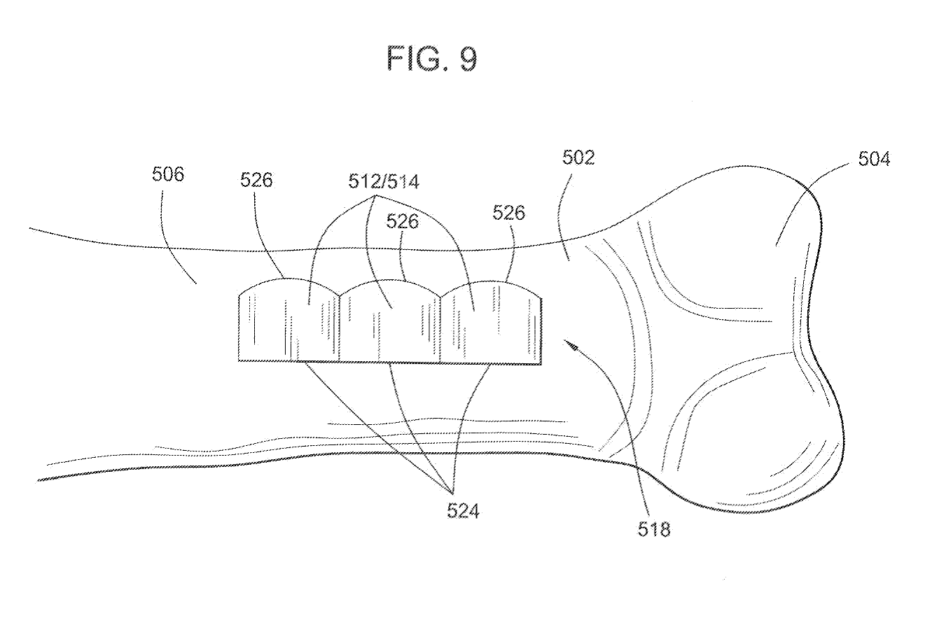

Described with respect to FIG. 9 is a method of producing intervertebral implants of the foregoing kind In accordance with the method, there is provided a donor bone 502 from which the implants can be harvested. The donor providing the donor bone can be from the same or different species as the intended recipient of the implant. The donor bone 502 is preferably a longer bone such as a femur, tibia, or humerus. Accordingly, the donor bone 502 has a condyle 504 or rounded distal end supported on a diaphysis 506 or the narrower shaft of the bone. Cut into the diaphysis and spaced from the condyle 504 can be a plurality of outlines 518 that correspond to the peripheral surface of the intervertebral implants 500 to be formed. At this step, one of the opposing first and second major surfaces 512, 514 corresponds to the outer surface of the donor bone 502 while the other surface remains intact inside the bone. By cutting the outlines parallel to the axis of the diaphysis, the majority of the bone tissue in the intervertebral implant including that exposed on the first and second surfaces can be cortical bone tissue. The outlines 518 are cut such that a plurality of repeating outlines are linearly aligned along and parallel to the axis of the diaphysis 506. The plurality of outlines is removed from the donor bone 502 and can be separated from each other to provide the implant. To shape the flat body of the intervertebral implant, the portions of the outline corresponding to the first, second, and peripheral surfaces can be planed. Apertures and/or grooves of the foregoing type can be disposed into the implant and osteogenic material can be added. An advantage of cutting the implants from along the diaphysis is that doing so makes greater use of the surface area of the bone by allowing more outlines to be cut. Additionally, cutting parallel along the diaphysis allows implants of a larger and more varied shape to be produced. Furthermore, cutting from the diaphysis allows greater variability of the height of the implant measured between the third straight edge 524 and fourth curved edge 514, including enabling heights greater than 2.0 cm.

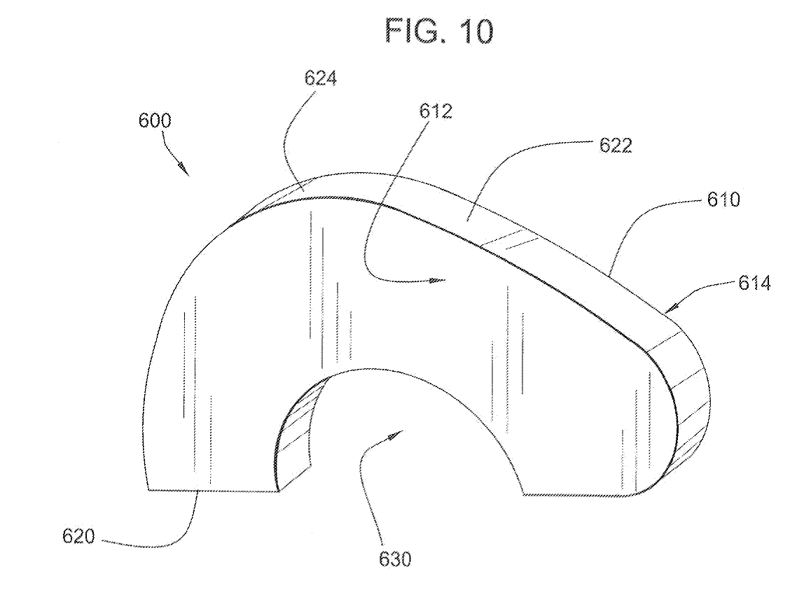

Referring to FIG. 10, there is illustrated another embodiment of an intervertebral implant 600 which is roughly shaped or outlined as a bow or question-mark. Particularly, the implant 600 can have a generally flat body 610 including a first surface 612, an opposing second surface 614, and a peripheral surface 618 interconnecting the first and second surfaces. To provide the bow or question-mark shape, the peripheral surface 618 can include a straight edge 620 and a curved edge 622 that bows outward and away from the straight edge. Moreover, the curved edge 620 can be distorted toward one half of the body 610. As illustrated, this causes the apex 624 of the curved edge 622 to be offset from the midpoint of the straight edge 620.

Disposed into the body 610 from the first straight edge toward the second curved edge 622 can be a cutout 630. The cutout 630 generally extends between and through the first and second surfaces 612, 614. Moreover, the cutout 630 can have any desired shaped and preferably has a rounded shape to conform generally to the shape of the curved edge 622. In various embodiments, the bone tissue proximate the curved edge 622 can be primarily cortical tissue while the bone tissue proximate the cutout 630 can be primarily cancellous tissue. The operation of forming the cutout 630 removes much of the cancellous tissue so that the remaining material of the implant 600 is primarily the biomechanically stronger cortical tissue. The cutout 630 can receive osteogenic material to promote bone growth and fusion of adjacent vertebrae. Moreover, in accordance with the foregoing embodiments, disposed into either or both of the first and second surfaces can be a plurality of grooves that can also prevent slipping of the inserted implant and/or receive osteogenic material.

The intervertebral implants described herein can be formed by any suitable forming operation. For example, a milling apparatus including a rotating end mill can be used to cut the implants from a donor bone and then to form the apertures and/or grooves. Additionally, the milling apparatus can be used with an end mill to plane the first and second surfaces so that the body is generally flat. To automate the process, the milling apparatus can be computer numerically controlled. In other embodiments, the intervertebral implants can be formed by traditional hand tools such as saw and/or osteotomes. After forming, the implants can be inserted freshly or can be stored in a frozen or freeze-dried state.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An intervertebral implant comprising:
a generally flat body of cortical bone cut parallel to the long axis of a bone diaphysis, the body having a first surface, an opposing second surface, and a peripheral surface between the opposing first and second surfaces;
a plurality of gull-wing shaped grooves disposed into the first surface;
at least one aperture disposed into the body from the first surface toward the second surface, the at least one aperture being disposed into the first surface on a non-perpendicular angle; and
osteogenic material received in the at least one aperture.

2. The intervertebral implant of claim 1, wherein the at least one aperture is generally cylindrical and extends through the second surface.

3. The intervertebral implant of claim 1, wherein the body is generally ‘D’-shaped and wherein the peripheral surface includes a first generally straight lateral edge, a second generally straight lateral edge parallel to the first edge, a third generally straight edge extending between the first and second lateral edges, and a fourth generally curved edge extending between the first and second lateral edges and opposing the third edge, the fourth curved edge being directed away from the third edge.

4. The intervertebral implant of claim 1, wherein the first surface and the second surface are non-parallel and inclined with respect to each other.

5. The intervertebral implant of claim 4, wherein the first and second surfaces are inclined on an angle of about 40° .

6. The intervertebral implant of claim 1, wherein the body has an average thickness between the first and second surfaces between about 0.5 cm to about 2.0 cm.

7. The intervertebral implant of claim 1, wherein the body has an average width between the first and second lateral edges of about 3 cm and an average length between the third edge and the apex of the fourth curved edge between about 1.3 cm to about 2.5 cm.

8. The intervertebral implant of claim 1, wherein the osteogenic material is selected from the group consisting of non-de-mineralized particular bone material, de-mineralized bone matrix, partially de-mineralized bone material, partially de-calcified bone material, AAA bone graft, or osteogenic growth factors including BMP.

9. The intervertebral implant of claim 1, wherein the material of the body is selected from allogeneic bone tissue and xenogeneic bone tissue.

10. The intervertebral implant of claim 1, wherein the at least one aperture includes a first section extending to 0.5 to 1.0 mm from the second surface and a second section disposed through the second surface to form a hole therein having a diameter of about 1.0 mm to about 2.0 mm.

11. An intervertebral implant comprising:
a body having a first surface, an opposing second surface, and a peripheral surface between the opposing first and second surfaces;
a plurality of gull-wing shaped grooves disposed into the first surface; and
osteogenic material received in the plurality of gull-wing shaped grooves.

12. The intervertebral implant of claim 11, wherein the body is generally ‘D’-shaped and wherein the peripheral surface including a first generally straight lateral edge, a second generally straight lateral edge parallel to the first edge, a third generally straight edge extending between the first and second lateral edges, and a fourth generally curved edge extending between the first and second lateral edges and opposing the third edge, the fourth curved edge being directed away from the third edge.

13. The intervertebral implant of claim 12, wherein the plurality of gull-wing shaped grooves each includes a first curve and a second curve both directed toward the fourth curved edge, the first and second curves intersecting approximately mid-width between the first and second lateral edges.
